(19) United States
(12) Patent Application Publication
(10) Pub. No.: US 2026/0315233 A1
(43) Pub. Date: Oct. 15, 2026

(54) BIPHASIC OSTEOCHRONDRAL GRAFTS

(71) Applicant: LifeNet Health, Virginia Beach, VA (US)

(72) Inventors: Alexander Huber, Virginia Beach, VA (US); Tak Lung Chang, Virginia Beach, VA (US); Jingsong Chen, Virginia Beach, VA (US)

(21) Appl. No.: 19/637,688

(22) Filed: Apr. 2, 2026

Related U.S. Application Data
(60) Provisional application No. 63/782,927, filed on Apr. 3, 2025.

(51) Int. Cl. A61F 2/30; A61L 27/36; A61L 27/50

(52) U.S. Cl. CPC A61F 2/30756; A61L 27/3654; A61L 27/3683; A61L 27/50; A61F 2002/30762

(57) ABSTRACT

A biphasic osteochondral graft for joint repair is provided. The graft includes two phases: a cartilaginous phase which includes at least a portion of a subchondral bone plate and calcified cartilage such that the at least a portion of the subchondral bone plate and the calcified cartilage are at least partially demineralized; and a substantially non-demineralized subchondral bone phase contacting and adjacent the subchondral bone plate. A modulus of the cartilaginous phase is less than a modulus of the substantially non-demineralized subchondral bone phase. Methods for using and apparatuses for preparing the biphasic osteochondral graft for joint repair are also provided.

### CROSS REFERENCE TO RELATED APPLICATIONS

This application is related to and claims the benefit of priority of U.S. Provisional Application No. 63/782,927 filed on Apr. 3, 2025, the entire contents of which are incorporated by reference herein for all purposes.

### FIELD OF THE INVENTION

This invention relates to engineered tissue grafts, especially those useful for the repair of articulating surfaces and associated tissues of synovial joints.

### BACKGROUND

Several problems exist with current grafts for the repair of articulating surfaces and associated tissues of synovial joints. Articulating joints have on their interior articulating surface, a soft cartilage layer which comprises a superficial zone, a middle zone, a deep zone, a tidemark, and calcified cartilage, and then subchondral bone. Therefore, it is desirable for grafts intended to repair such joints to have soft cartilage-inducing tissue on the articulating surface of the joint and also to have a bone inducing tissue opposite the cartilage surface, to mimic the tissue structure in such joints. However, when such tissues are used for repair of joints, i.e., the above structure of a soft cartilage layer which includes a superficial zone, a middle zone, a deep zone, a tidemark, and optionally a portion of calcified cartilage, the cartilage layer tends to slough off, and not provide an effective repair. If the soft cartilage-superficial zone, middle zone, deep zone, tidemark, and optionally the calcified cartilage are removed from such grafts, with the objective being to provide a more robust surface for the repair, the remaining calcified cartilage (if any) and bone tissue tends to induce formation of bone in the articulating surface of the joint, rather than the desired cartilage induction.

Thus, a biphasic graft for joint repair that is capable of inducing cartilage growth on its articulating surface and bone growth on its opposite surface, while ensuring zone integrity without separation of cartilage and bone is desired.

### SUMMARY

The inventors have solved this problem by producing a biphasic graft for joint repair in which the soft cartilage, which may comprise the superficial zone, middle zone, deep zone, tidemark, and optionally a portion of the calcified cartilage are removed from such grafts, and then the grafts are processed to provide a suitable compartment area which induces cartilage growth and a compartment area opposite thereof that induces bone growth. A compartment area is a section of the graft.

A biphasic osteochondral graft for joint repair is provided. The graft comprises a cartilage-like phase (cartilaginous phase). The cartilage-like phase comprises at least a portion of a subchondral bone plate and calcified cartilage. Also, in the cartilage-like phase, at least a portion of the subchondral bone plate and the calcified cartilage are at least partially demineralized. Another phase of the biphasic graft comprises a substantially non-demineralized subchondral bone phase contacting and adjacent the subchondral bone plate. Importantly, a modulus of the cartilage-like phase (cartilaginous phase) is less than a modulus of the substantially non-demineralized subchondral bone phase.

A method of preparing the biphasic osteochondral bone graft is also provided. The method may comprise the following steps. A source of the biphasic osteochondral graft may be obtained from a donor. The source may be disarticulated to provide a section of osteochondral tissue. The section of osteochondral tissue may comprise soft cartilage, calcified cartilage, a subchondral bone plate, and subchondral bone. At least a portion of the soft cartilage may be removed. Portions of the subchondral bone plate and/or the calcified cartilage may be at least partially demineralized with a demineralization solution to provide the cartilage-like (cartilaginous) phase. At least a portion of the subchondral bone plate and the subchondral bone may be exposed to a buffering solution to mitigate, minimize, or reduce the effects of the demineralization solution, to provide the substantially non-demineralized bone phase. The exposure to the buffering solution may be done during or after the treatment with the demineralization solution. The buffering solution reduces, minimizes, or mitigates the effects of the demineralization solution.

The resulting biphasic graft has a property in which the modulus of the cartilage-like (cartilaginous) phase is less than a modulus of the original (non-demineralized) calcified cartilage and is also less than a modulus of the at least a portion of the subchondral bone plate prior to the demineralization step, i.e., the non-demineralized subchondral bone plate. In addition, the modulus of the substantially non-demineralized bone phase is about the same as a modulus of any of the subchondral bone plate and/or the subchondral bone prior to the demineralization step, i.e., the non-demineralized subchondral bone plate and the non-demineralized subchondral bone from which the graft was prepared.

Also provided is an apparatus for performing the above method. The apparatus comprises the following components. A first chamber and a second chamber. The first chamber is constructed and arranged to contain the demineralization solution and/or the growth factor removal solution; and the cartilage-like (cartilaginous) phase. The second chamber is constructed and arranged to contain the buffering solution, and substantially non-demineralized subchondral bone phase. The first chamber and the second chamber are not in fluid communication. The chamber's solutions may be subject to continuous or intermittent flow to increase flow through and exchange within the tissue sample during processing. The flow therefore may be laminar or turbulent or a combination thereof.

A method of repairing an osteochondral defect in a subject in need thereof is provided. The method comprises the following steps. Implanting at least one biphasic osteochondral graft into the osteochondral defect such that the substantially non-demineralized subchondral bone phase of the biphasic osteochondral graft is in an interior of the defect and the cartilage-like (cartilaginous phase) of the biphasic osteochondral graft is on an exterior of the defect. Then, sealing the implanted biphasic osteochondral graft into recipient tissue.

### BRIEF DESCRIPTION OF THE DRAWINGS

FIG. 1 shows the anatomy of an articulating joint.

FIG. 2 shows a diagram of a section of a source of the biphasic osteochondral graft for joint repair.

FIG. 3 shows a photo of an embodiment of a biphasic osteochondral graft for joint repair.

FIG. 4 shows a diagram of a section of an embodiment of a biphasic osteochondral graft for joint repair.

FIG. 5 shows a diagram of a section of an embodiment of a biphasic osteochondral graft for joint repair showing cartilage factors in the cartilaginous phase and bone factors in the bone phase.

FIG. 6 shows a process diagram of an embodiment of a method of preparing a biphasic osteochondral graft for joint repair.

FIG. 7 shows a process diagram of another embodiment of a method of preparing a biphasic osteochondral graft for joint repair.

FIG. 8 shows a diagram of an embodiment of an apparatus for preparing a biphasic osteochondral graft for joint repair.

FIG. 9 shows a diagram of another embodiment of an apparatus for preparing a biphasic osteochondral graft for joint repair.

FIG. 10 shows a photograph of another embodiment of an apparatus for preparing a biphasic osteochondral graft for joint repair.

FIG. 11 shows photographs of lyophilized and hydrated biphasic osteochondral grafts for joint repair.

FIG. 12 shows photographs of a biphasic osteochondral graft for joint repair being rehydrated and then placed in an osteochondral defect.

FIG. 13 shows an example of the biphasic osteochondral grafts for joint repair as used in a “snowman procedure.”

FIG. 14 shows Masson's Trichrome on a resin-embedded biphasic osteochondral graft sample cross-section.

FIG. 15 shows Hematoxylin & Eosin and Collagen type II immunostained articular surface of the cartilaginous phase of a biphasic graft for joint repair.

FIG. 16 shows stress-strain curves of non-demineralized and demineralized cartilaginous phases compared to articular cartilage.

FIG. 17 shows Colorimetric quantification of sGAG (Blyscan assay) and BMP-2 ELISA on solubilized tissue samples.

FIG. 18 shows assessment of material's osteoinductivity via alkaline phosphatase activity in C2C12 musculoskeletal progenitor cells in conditioned media.

FIG. 19 shows assessment of material's chondrogenic potential through culture of bone marrow-MSCs on “cartilage phase.”

FIG. 20 shows how the cartilaginous phase supports bone marrow-MSCs chondrogenic differentiation and cartilaginous neotissue formation in vitro.

FIG. 21 shows how the cartilaginous phase supports bone marrow-MSCs chondrogenic differentiation and cartilaginous neotissue formation in vitro indicated by presence of GAGs (Safranin O).

FIG. 22 shows how the cartilaginous phase supports bone marrow-MSCs chondrogenic differentiation and cartilaginous neotissue formation in vitro indicated by presence of Collagen II (ICC).

### DETAILED DESCRIPTION

An osteochondral defect is a defect in an articulating joint that extends from the superficial zone through the subchondral bone. Thus, a graft to repair this defect needs to be able to induce formation of at least the cartilage tissue as well as the subchondral bone tissue.

The inventors have prepared such a graft that has the ability to induce growth of cartilage tissue at the articulating surface as well as the subchondral bone. Unlike previous attempts to prepare such grafts, the superficial surface on the articulating surface of the joint does not tend to slough off, and also has the ability to induce growth of the softer (lower modulus) but still strong cartilage and the end opposite the superficial surface is harder (higher modulus) and induces the growth of bone.

Biphasic as used herein means a single unitary construct that has at least two phases that are different from each other. The difference may be in tissue type and/or in physical properties, such as the modulus. The different phases may have a distinct boundary between them, or the two phases may have a less distinct boundary such the tissue type and/or physical properties form a continuum of tissue types and/or physical properties. The single construct having two phases may be a combination of these two boundaries, i.e. the single construct may have a distinct boundary between the two phases. The single construct may have a continuum with a range of properties between two or more different tissue types and/or physical properties for example. The biphasic graft construct may be formed by assembling two or more separate pieces or may be a single piece. The biphasic graft may be a single continuous construct that has at least two phases that are different from each other. In other words, the interface between the two or more phases may comprise an abrupt change of properties or may comprise a smoother transition from one phase to the next, where the interface has intermediate properties. In this case, it is understood that the difference in modulus between the two phases is the largest difference.

Cartilage-like phase, “cartilage phase”, and cartilaginous phase as used herein are interchangeable. As used here, these terms mean a phase that has the ability to induce the growth of cartilage. This phase may be derived from cartilage. This phase may not be derived from cartilage. This phase has the properties of being soft-elastic, having a modulus less than that of the subchondral bone phase, able to support cartilaginous neotissue formation, and is non-osteoinductive.

Subchondral bone phase, or bone phase or “bone phase” as used herein means that this phase may or may not be derived from bone or subchondral bone but has the mechanical profile of bone especially having a modulus higher than that of the cartilaginous phase. The subchondral bone phase supports osteointegration and remodeling. It is osteoconductive, and/or osteoinductive.

Tidemark as used herein describes the anatomical border between the calcified cartilage and the soft cartilage.

Cement line as used herein describes the anatomical border between the calcified cartilage and the subchondral bone.

Substantially non-demineralized means that at least 80, 85, 90, 91, 92, 93, 93.5, 94, 94.5, 95, 95.5, 96, 96.5, 97, 97.5, 98, 98.5, 99, 99.5, or 100% by weight of the original mineral content remains. The mineral may include calcium. The mineral may comprise calcium and phosphate. The mineral may comprise hydroxyapatite [Ca₁₀ (PO₄)₆ (OH₂)].

Substantially demineralized means that at least 80, 85, 90, 91, 92, 93, 93.5, 94, 94.5, 95, 95.5, 96, 96.5, 97, 97.5, 95, 95.5, 96, 96.5, 97, 97.5, 98,98.5, 99, 99.5, or 100% by weight of the original mineral content has been removed. The mineral may include calcium. The mineral may comprise calcium and phosphate. The mineral may comprise hydroxyapatite [Ca₁₀(PO4)₆ (OH₂)]. The demineralization may not be uniform across a cross section of the sample. The demineralization may be higher on the outside of the sample and lower on the inside of the sample.

As used herein, the term “portion of” or “a portion” or “portion” means any non-zero amount of or, for example, but not limited to 1%, 5%, 10%, 15%, 20%, 25%, 30%, 40%, 50%, 60%, 70%, 80%, 90%, 95%, 98%, 99%, or up to 100% of the material that is described. The percents may be by weight or by volume.

As used herein, the modulus referred to is the unconstrained compressive modulus, measured as described in Kabir et al.; Cartilage 2021, Vol. 13 (Suppl 2) 427S-437S, the contents of which are incorporated by reference herein.

### Joint and Source of Biphasic Graft for Joint Repair

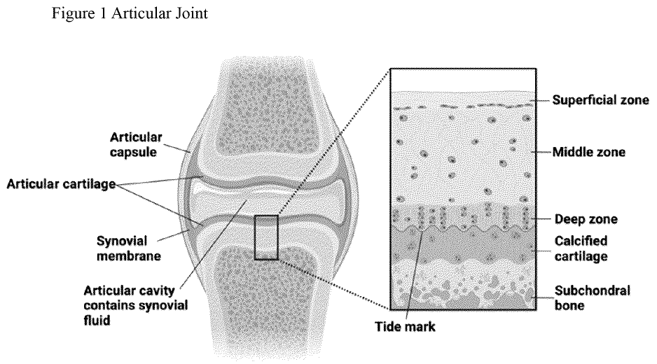

An articulating joint is shown in FIG. 1. As shown in FIG. 1, the articular capsule includes a synovial membrane, which encloses the articular cartilage, and holds the synovial fluid, which serves to lubricate articular cartilage which forms the moving surfaces of the joint. Looking more deeply at the anatomy of the layers of tissue which are connected to and comprise the articular cartilage, FIG. 1 shows that the surface of the articular cartilage has a superficial zone, a middle zone, a deep zone, calcified cartilage, and then subchondral bone, or the subchondral bone plate. The demarcation between the deep zone and the calcified cartilage is the tidemark, as shown in FIG. 1. The demarcation between the calcified cartilage and the subchondral bone is the cement line.

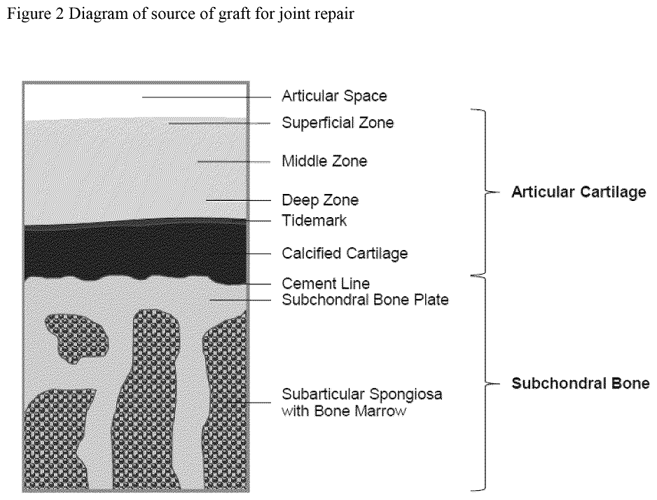

FIG. 2 shows a diagram of a source of the biphasic graft for joint repair, prior to being subjected to the methods of preparing the biphasic osteochondral graft for joint repair described herein. As seen in FIG. 2, the source of the biphasic osteochondral graft for joint repair may be from a whole or a section of an articular joint. The source of the biphasic osteochondral graft for joint repair may comprise articular cartilage and subchondral bone. The articular cartilage, starting at the articular space may comprise a superficial zone, a middle zone, a deep zone and calcified cartilage. The interface between the calcified cartilage and the deep zone is the tidemark. The subchondral bone comprises the subchondral bone plate and also the subarticular spongiosa with bone marrow, as shown in FIG. 2. The cement line comprises the interface between the calcified cartilage and the subchondral bone plate, as shown in FIG. 2.

### Biphasic Osteochondral Graft

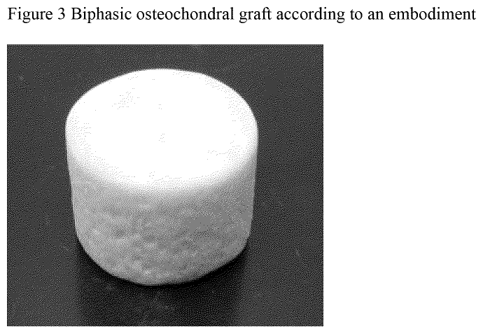

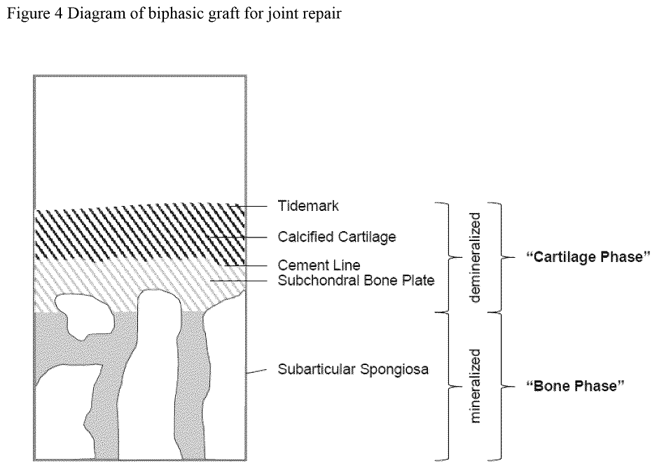

A biphasic osteochondral graft for joint repair is provided. FIG. 3 shows a photograph of a biphasic osteochondral graft as described herein. FIG. 4 shows a diagram of the biphasic osteochondral graft showing the components of the biphasic graft. The biphasic graft comprises a cartilage-like (cartilaginous phase or “cartilage phase”) and a “bone phase”. As seen in FIG. 4, the “cartilage phase” is at least partially demineralized and the “bone phase” is substantially non-demineralized. The cartilaginous phase comprises at least a portion of a subchondral bone plate and calcified cartilage. Also in the cartilaginous phase, at least a portion of the subchondral bone plate and the calcified cartilage are at least partially demineralized. Another phase of the biphasic graft comprises a substantially non-demineralized subchondral bone phase contacting and adjacent the subchondral bone plate. Importantly, a modulus of the cartilage-like phase (cartilaginous phase) is less than a modulus of the substantially non-demineralized subchondral bone phase.

The biphasic osteochondral graft may be lyophilized. The biphasic osteochondral graft may be a complex, tissue-derived and tailored for a specific site. The biphasic osteochondral graft may comprise collagens, including for example, collagen I, collagen II, and/or collagen III. The biphasic osteochondral graft may comprise growth factors such as platelet-derived growth factor (PDGF), epidermal growth factor (EGF), keratinocyte growth factor (KGF), insulin-growth factor 1 (IGF-1), transforming growth factor β (TGF-β), fibroblastic growth factors (FGFs), and/or bone morphogenetic protein (BMP). The biphasic osteochondral graft may further comprise glycosaminoglycans, such as hyaluronic acid, and extracellular vesicles (EVs).

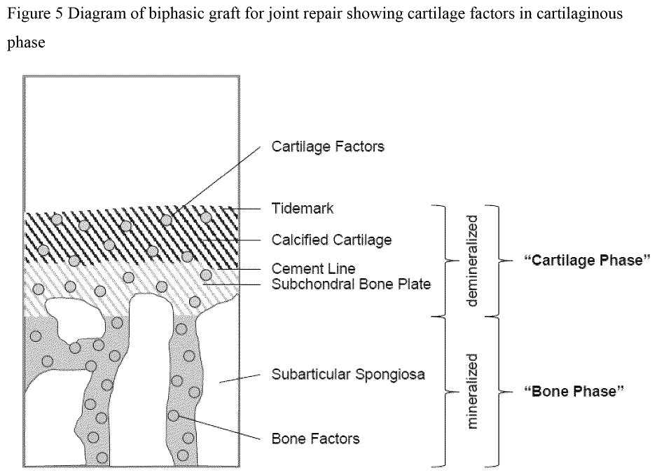

As shown in FIG. 5, the “cartilage phase” or cartilaginous phase may comprise cartilage growth factors and the “bone phase” may comprise bone growth factors. Non-limiting examples of cartilage growth factors are insulin-growth factor I (IGF-I), transforming growth factor β (TGF-β), fibroblastic growth factors (FGFs), bone morphogenic proteins (BMP) and growth and differentiation factor 5 (GDF-5). Examples of FGFs include FGF-2, FGF-18, any truncated or modified forms and mixtures thereof. Examples of BMPs include BMP-2, BMP-7, any truncated or modified forms and mixtures thereof. Other bone-associated factors include glycosaminoglycans and extracellular vesicles (EVs). These cartilage growth factors may already be present in the cartilage phase of the graft or they may be added to the cartilage phase of the graft during the preparation of the graft. The cartilage growth factors may be a combination of already present cartilage growth factors and cartilage growth factors added during preparation of the graft. According to some embodiments, these added cartilage growth factors may be extracted from the same donor as the graft. According to some other embodiments, these added cartilage growth factors may be extracted from a different donor from the graft donor. According to yet another embodiment, these added cartilage growth factors may be extracted from the recipient of the graft.

Non-limiting examples of bone growth factors are bone morphogenetic proteins (BMPs), transforming growth factor beta (TGF-ß), insulin-like growth factors I and II (IGF-I and IGF-II), platelet derived growth factor (PDGF) and basic and acidic fibroblast growth factor (bFGF and aFGF) and mixtures thereof. Others include BMP, PDGF, FGF, VEGF, TGF, insulin, among others. Examples of BMPs include BMP-2, BMP-3, BMP-4, BMP-5, BMP-6, BMP-7, BMP-8, BMP-9, BMP-10, BMP-11, BMP-12, BMP-13, BMP-14, BMP-15, any truncated or modified forms of BMP-2, BMP-3, BMP-4, BMP-5, BMP-6, BMP-7, BMP-8, BMP-9, BMP-10, BMP-11, BMP-12, BMP-13, BMP-14, or BMP-15, and mixtures thereof. These bone growth factors may already be present in the bone phase of the graft or they may be added to the bone phase of the graft during the preparation of the graft. The bone growth factors may be a combination of already present bone growth factors and bone growth factors added during preparation of the graft. According to some embodiments, these added bone growth factors may be extracted from the same donor as the graft. According to some other embodiments, these added bone growth factors may be extracted from a different donor from the graft donor. According to yet another embodiment, these added bone growth factors may be extracted from the recipient of the graft. According to some embodiments, growth factors may be added to the bone phase according to the procedures described in International application No. WO2024/086171 A1, the entire contents of which are incorporated by reference herein for all purposes. According to some embodiments, growth factors may be extracted according to the procedures described in International application No. WO 2024/086171 A1.

The cartilaginous phase of the biphasic osteochondral graft may comprise the calcified cartilage layer, wherein the calcified cartilage is at least partially demineralized. For example, the demineralized calcified cartilage may comprise at most 20, 19, 18, 17, 16, 15, 14, 13, 12, 11, 10, 9, 8, 7, 6, 5, 4, 3, 2, 1, 0.5, 0.1, 0.01 wt % calcium based on the dry weight of the calcified cartilage layer. The at least partially demineralized calcified cartilage may comprise at most 90, 85, 80, 70, 60, 50, 40, 30, 20, 10, 9, 8, 7, 6, 5, 1, 0.5, 01, 0.05, 0.1% by weight of calcium compared to the calcium content of a non-demineralized calcified cartilage layer. The cartilaginous phase may comprise a cement line, wherein the cement line may be at least partially demineralized. For example, the cement line may comprise at most 15, 14, 13, 12, 11, 10, 9, 8, 7, 6, 5, 4, 3, 2, 1, 0.5, 0.1, 0.01 wt % calcium based on the dry weight of the cement line. The demineralized cement line may comprise at most 90, 80, 70, 60, 50, 40, 30, 20, 10, 5, 1, 0.5, 01, 0.05, 0.1% by weight of calcium compared to the calcium content of a non-demineralized cement line.

The cartilaginous phase of the biphasic osteochondral graft may have a modulus of from 1 kPa to 10 MPa. For example the modulus of cartilaginous phase of the biphasic osteochondral graft may be at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 kPa, 1MPa, 1.1, 1.2, 1.3, 1.4, 1.5, 1.6, 1.7, 1.8,1.9 MPa. For example, the modulus of cartilaginous phase of the biphasic osteochondral graft may be at most 10, 9, 8, 7, 6, 5, 4, 3, 2, 1.9, 1.8, 1.7, 1.6 1.5, 1.4, 1.3, 1.2, 1.1, 1 MPa, 900 kPa, 890, 880, 870, 860, 850, 840, 830, 820, 810, 800, 790, 780, 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10, 9, 8, 7, 6, 5, 4, 3, 2, or at most 1 kPa. Thus, the modulus of the cartilaginous phase of the biphasic osteochondral graft may be reduced by the method of treatment of the source of the graft by at least 1000%, 900%, 800%, 700%, 600%, 500%, 400%, 300%, 200%, 100%, 90%, 80%, 70%, 60%, 50, 40%, 30%, 20% or at least by 10% compared to a modulus of the portion of the source of the graft corresponding to the cartilaginous phase prior to the demineralization step.

The substantially non-demineralized subchondral bone phase of the biphasic osteochondral graft may comprise more than 81, 82, 83, 84, 85, 86, 87, 88, 89, 90, 91, 91.5, 92, 92.5, 93, 93.5, 94, 94.5, 95, 95.5, 96, 96.5, 97, 97.5, 98, 98.5, 99, 99.5, or 100% of the mineral content of the sample from which the subchondral phase is prepared. The mineral may comprise calcium. The mineral may comprise calcium and phosphate. The mineral may comprise hydroxyapatite [Ca₁₀ (PO₄)₆ (OH₂)]. The demineralization may not be uniform across a cross section of the sample. The demineralization may be higher on the outside of the sample and lower on the inside of the sample. The substantially non-demineralized bone phase may have had at least a portion of bone marrow removed. For example, at least 5, 10, 15, 20, 30, 35, 40 50, 60, 73, 80, 90, 95, 98, 99, or 100 % by weight of the bone marrow may have been removed. For example, at most 5, 10, 15, 20, 30, 35, 40, 50, 60, 73, 80, 90, 95, 98, 99, or 100 % by weight of the bone marrow may have been removed.

According to an embodiment, the bone phase may remain viable. The bone phase may remain viable after removal of at least a portion of the bone marrow.

According to an embodiment the cartilaginous phase may be viable. According to another embodiment, the cartilaginous phase may not be viable. According to yet another embodiment, the cartilaginous phase may have few to no viable cells.

As used herein, viable means that at least a portion of the cells present in the bone phase and/or the cartilaginous phase are capable of at least one of growing, metabolizing, proliferating, engaging in cell processes.

According to another embodiment, holes, also referred to as “drill holes” may be present in the bone phase. The holes are not those that may be naturally present in the bone phase and may be introduced by drilling for example. The holes may facilitate cell ingrowth and bony fusion. They may start from the bottom part, i.e., the end of the of the bone phase of biphasic graft that is the furthest from the interface between the bone phase and cartilaginous phase. The holes may end at or in the interface of the bone phase and cartilaginous phase, while not penetrating through the surface of the graft, other than at their entry point at the end of the of the bone phase of biphasic graft that is the furthest from the interface between the bone phase and cartilaginous phase.

According to an embodiment, the biphasic osteochondral graft for joint repair may be cryopreserved.

The cartilaginous phase of the biphasic osteochondral graft may comprise less than 45, or less than 35, or less than 30, or less than 25, or less than 15 pg/mg dry tissue of bone morphogenetic protein 2 (BMP-2). The substantially non-demineralized subchondral bone phase of the biphasic osteochondral graft may comprise 10, or 15, or 25, or 35, or 45 pg/mg dry tissue or more of bone morphogenetic protein 2 (BMP-2). The cartilaginous phase of the biphasic osteochondral graft may comprise 2 μg/mg dry tissue or more of glycosaminoglycans (sGAG). The cartilaginous phase of the biphasic osteochondral graft may comprise 3, 4, 5, 6, 7, 8, 9, 10, 15, 20, 30, 40, 50, 60, 70, 80, 100μg/mg dry tissue or more of glycosaminoglycans (sGAG). The sGAGs may or may not be sulfated. Nonlimiting examples of sGAGs are hyaluronic acid (HA), heparin/heparan sulfate (HSGAGs), chondroitin sulfate/dermatan sulfate (CSGAGs), keratan sulfate. These sGAGs may be either added, naturally present, or a combination. They may be present alone or in combination.

The substantially non-demineralized subchondral bone phase of the biphasic osteochondral graft may comprise less than 5, 4, 3, 2, 1, 0.5, 0.1, or less than 0.05 μg/mg dry tissue of glycosaminoglycans (sGAG). Nonlimiting examples of sGAGs are hyaluronic acid (HA), heparin/heparan sulfate (HSGAGs), chondroitin sulfate/dermatan sulfate (CSGAGs), keratan sulfate. These sGAGs may be either added, naturally present, or a combination. They may be present alone or in combination.

The substantially non-demineralized subchondral bone phase of the biphasic osteochondral graft may have a modulus of from 10 MPa to 2 GPa (2000 MPa). For example the modulus of the non-demineralized subchondral bone phase of the biphasic osteochondral graft may be at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 MPa, 1 GPa, 1.1, 1.2, 1.3, 1.4, 1.5, 1.6, 1.7, 1.8, 1.9 GPa. For example, the modulus of the non-demineralized subchondral bone phase of the biphasic osteochondral graft may be at most 2, 1.9, 1.8, 1.7, 1.6 1.5, 1.4, 1.3, 1.2, 1.1, 1 GPa, 900 MPa, 890, 880, 870, 860, 850, 840, 830, 820, 810, 800, 790, 780, 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, or at most 20 MPa.

The modulus of the non-demineralized bone phase may be at least 100%, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61 or 60 % of the modulus of the part of the sample of the source from which the graft is prepared that corresponds to the non-demineralized bone phase after the demineralization step.

According to an embodiment, the biphasic osteochondral graft may be isolated from, i.e., the source of the graft may be articulating surfaces from synovial joints, including, but not limited to those associated with the femur (i.e. condyle, femoral head), tibia, patella, fibula, humerus, radius, ulna, talus, calcaneus tarsal and metatarsal bones, carpal and metacarpal bones, phalanges, and segments thereof (e.g., hemicondyles, hemiplateaus), and/or combinations of any two or more of the foregoing. According to an embodiment, the biphasic osteochondral graft may be isolated from non-synovial joints, including, but not limited to those associated with the ribs, the spinal column, including the intervertebral discs, and the nose.

The biphasic osteochondral graft for joint repair may be capable of being shaped. Non-limiting examples of suitable shaping techniques or instruments include using a blade, drill, mallet or buffs including automated tools using these principles.

The biphasic osteochondral graft may be an allograft, autograft or xenograft. The biphasic osteochondral graft may be isolated from an animal or human. The animals may be vertebrates or invertebrates. The animal may be selected from ovine, bovine, canine, caprine, piscine, shark, or porcine.

### Method of Preparing the Biphasic Osteochondral Graft

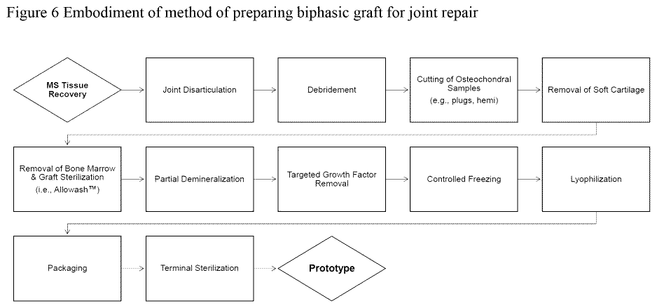

A method of preparing the biphasic osteochondral graft is also provided. FIG. 6 shows a flow diagram of an embodiment of the method of preparing the biphasic osteochondral graft according to the principles of the present invention. As depicted in FIG. 6, the method comprises the following steps. The order of these steps may be changed as appropriate. First, obtaining a source of the biphasic osteochondral graft from a donor. Then, the source may be disarticulated to provide a section of osteochondral tissue. The section of osteochondral tissue may comprise soft cartilage, calcified cartilage, a subchondral bone plate, and subchondral bone. At least a portion of the soft cartilage may be removed. The removal may be done by way of debridement, using a suitable sharp instrument or instruments. At least a portion of the subchondral bone plate and the calcified cartilage may be at least partially demineralized with a demineralization solution to provide the cartilage-like (cartilaginous) phase. At least a portion of the subchondral bone plate and the subchondral bone may be exposed to (treated) with a buffering solution or a saline solution or water for example, to provide the substantially non-demineralized bone phase. The buffering solution may minimize, mitigate, or reduce the demineralization effects of the demineralization solution.

The resulting biphasic graft has a property in which the modulus of the cartilage-like (cartilaginous) phase is less than a modulus of the calcified cartilage and the at least a portion of the subchondral bone plate prior to the demineralization step. In addition, the modulus of the substantially non-demineralized bone phase is about the same as a modulus of any of the subchondral bone plate and the subchondral bone prior to the demineralization step.

The modulus of the calcified cartilage phase prior to the demineralization step may be from 10 MPa to 2 GPa.

The modulus of the calcified cartilage portion of the cartilaginous phase after the demineralization step may be from 20 kPa to 10 MPa (10,000 kPa).

The modulus of the at least a portion of the subchondral bone plate prior to the demineralization step may be from 0.5 to 4 GPa.

The removal of the at least a portion of the soft cartilage may be done by debridement, by cutting, grinding, or scraping.

The section of osteochondral tissue may comprise a tidemark and the tidemark may be at least partially demineralized such that the cartilaginous phase comprises the at least partially demineralized tidemark; and the modulus of the cartilaginous-like phase is less than the modulus of the tidemark, the calcified cartilage, and the at least a portion of the subchondral bone plate prior to the demineralization step. The section of osteochondral tissue may comprise a cement line and the cement line may be at least partially demineralized such that the cartilaginous phase comprises the at least partially demineralized cement line; and wherein the modulus of the cartilaginous phase is less than the modulus of the cement line, the calcified cartilage, and the at least a portion of the subchondral bone plate prior to the demineralization step.

The modulus of the tidemark prior to the demineralization step may be from 20 kPa to 10 MPa (10,000 kPa).

The modulus of the at least partially demineralized tidemark may be from 0.2 kPa to less than 10 MPa. The modulus of the at least partially demineralized tidemark may be reduced by at least 1000%, 900%, 800%, 700%, 600%, 500%, 400%, 300%, 200%, 100%, 90%, 80%, 70%, 60%, 50, 40%, 30%, 20% or at least by 10% compared to a modulus of the source of the tidemark prior to the demineralization step.

The modulus of the cement line prior to the demineralization step may be from 0.5 to 7.5 GPa.

The modulus of the at least partially demineralized cement line may be from 5 Pa to less than 7.5 GPa. The modulus of the at least partially demineralized tidemark may be from 0.2 kPa to less than 10 MPa. The modulus of the at least partially demineralized cement line may be reduced by at least 1000%, 900%, 800%, 700%, 600%, 500%, 400%, 300%, 200%, 100%, 90%, 80%, 70%, 60%, 50, 40%, 30%, 20% or at least by 10% compared to a modulus of the source of the cement line prior to the demineralization step.

The demineralization solution may be an acid. For example, 0.5 to 0.6 N hydrochloric acid, or alternatively 0.6 to 1.0 N hydrochloric acid may be used. Other suitable acids are formic acid, acetic acid, ethylene diamine, tetraacetic acid, sulfuric acid, citric acid, or nitric acids. The concentration and type of acid may be selected depending on such factors as the temperature of demineralization, the time of demineralization and the desired extent of demineralization. Suitable pH is from 0 to 4, for example. The time for demineralization may be from about 1 minute to about 8 hours, at 25 °C to 50 °C., or from 25 °C to 37 °C. Other time periods may be from 5 minutes to 4 hours, or from 10 minutes to 1 hour. The time for demineralization depends on the amount of demineralization to be achieved as well as the temperature, and time of the demineralization process and the concentration of the acid solution. The flow rate of the acid, including such parameters as the Reynolds number may also affect the rate of demineralization.

The buffering solution, which is used to prevent or stop (mitigate) or minimize demineralization of a remaining portion of the subchondral bone plate and the underlying subchondral bone which was not exposed to or treated with the demineralization solution may comprise phosphate buffered saline (PBS); [tris(hydroxymethyl)methylamino]propanesulfonic acid) (TAPS); tris(hydroxymethyl)aminomethane, or 2-amino-2-(hydroxymethyl)propane-1,3-diol) (Tris); 2-(bis(2-hydroxyethyl)amino)acetic acid (Bicine); N-[tris(hydroxymethyl)methyl]glycine (Tricine); 3-[N-tris(hydroxymethyl)methylamino]-2-hydroxypropanesulfonic acid (TAPSO); 4-(2-hydroxyethyl)-1-piperazineethanesulfonic acid; (HEPES); 2-[[1,3-dihydroxy-2-(hydroxymethyl)propan-2-yl]amino]ethanesulfonic acid (TES); 3-(N-morpholino)propanesulfonic acid (MOPS); piperazine-N, N′-bis(2-ethanesulfonic acid) (PIPES); dimethylarsenic acid (Cacodylate); 2-(N-morpholino)ethanesulfonic acid (MES). The buffering solution may maintain a pH of about 5-9, or 6-9, or 7-9, or 8-9, or 5-8, or 5-7, or 5-6, or 7-8, or 7-9, or 6-7, or 6-8, or about 6 or about 7 or about 8.

The method may further comprise removing osteoinductive factors including bone morphogenetic protein 2 (BMP-2) from the cartilaginous phase with a growth factor removal solution, without substantially removing osteoinductive factors from the substantially non-demineralized bone phase, using chaotropic agents and/or detergents. For example, 0.5-5.0 M Guanidine HCl in deionized water, saline, or other suitable aqueous media may be used. Other chaotropic agents, detergents, and surfactants may include n-butanol, ethanol, guanidinium chloride, lithium perchlorate, lithium acetate, magnesium chloride, phenol, 2-propanol, sodium dodecyl sulfate, Triton X-100, thiourea, or urea. Non-limiting examples of suitable detergents include zwitterionic, non-ionic, anionic, or cationic detergents. The detergent solution may be followed by or combined with an enzyme. The time for osteoinductive factor removal may be from about 1 minute to about 8 hours, at 25 °C to 50 °C., or from 25 °C to 37 °C. Other time periods may be from 5 minutes to 4 hours, or from 10 minutes to 1 hour.

The method may further comprise removing bone marrow from the graft. The method may further comprise removing bone marrow from the bone phase of the graft. Bone marrow may be removed by methods such as but not limited to detergent washes, enzymatic digestion, and/or freeze-thaw cycles. Non-limiting examples of suitable detergents include zwitterionic, non-ionic, anionic, or cationic detergents. According to some embodiments, the detergent may be combined with short-chain alcohols, such as ethanol or propanol. Non-limiting examples of suitable detergents are polyoxyethylene alcohols, polyethylene glycol p-isooctylphenylethers, polyoxyethylene nonylphenol, and polyoxyethylene sorbitol esters. Allowash® brand solution (LifeNet Health) may be used. A combination of polyoxyethylene-4-lauryl, octylphenol-ethyleneoxide and poly(ethylene glycol)p-nonyl-phenyl-ether may be used. The bone marrow may be removed at any point in the method, i.e., before or after removing the soft cartilage, before or after the demineralization step.

Tissue processing may utilize a processing chamber that separates the cartilaginous phase from the bone phase and selective exposure of processing solutions.

The method may further comprise freezing (cryopreserving) the source of the biphasic osteochondral graft. The freezing temperature may be 0 °C or below. The freezing temperature may be about −15, −25, −80, −100, −125, −150, −175, −196, or −200 °C. The source may be frozen before, during or after the source is obtained from a donor. The source may be disarticulated before, during or after the disarticulation step.

The method may further comprise freezing (cryopreserving) the biphasic osteochondral graft. The freezing temperature may be about −15, −25, −80, −100, −125, −150, −175, −196, or 200 °C or lower.

The method may further comprise lyophilization of the biphasic osteochondral graft. Lyophilization may be done at temperatures 25 °C or below. The lyophilization temperature may be about 25, 20, 10, 0, −5, −15, −25, −75, −100, −125, −150, −175, −196, or −200 °C or lower. Lyophilization is carried out at sub-ambient pressures. Lyophilization is advantageously carried out in stages. A suitable lyophilization procedure may be 60 minutes at −5 °C and 350 mTorr (47 Pa), 2000 minutes at 0 °C and 350 m Torr (47 Pa) and 500 minutes at 22 °C and 50 mTorr (7 Pa).

The method may further comprise sterilizing at least a portion of the biphasic osteochondral graft. For example, the cartilaginous phase may be sterilized. According to another embodiment, the bone phase may be sterilized. In another embodiment, the entire biphasic osteochondral graft may be sterilized. Non-limiting examples of sterilization methods are radiation, such as gamma radiation, e-beam radiation, UV radiation. Other non-limiting examples of suitable sterilization methods are autoclave or pasteurization or supercritical CO₂.

FIG. 6 shows a flowchart of one method of preparing a biphasic osteochondral graft. As shown in FIG. 6, the method may comprise a step of recovering or obtaining the tissue from a donor. The tissue may be a joint as described above. The joint may be frozen and then processed. The joint is then disarticulated. The joint may be frozen after disarticulation. The joint may then be debrided. The debrided joint may then have one or more samples cut therefrom. The samples may be in the form of plugs and/or be the entire hemi joint and include soft cartilage, calcified cartilage, a subchondral bone plate, and subchondral bone. The plugs may have a circular cross section, or a square cross section, or a rectangular cross section, or any ovoid or polygonal cross section, or any combination thereof. At least a portion of the soft cartilage may then be removed. The portion may be up to 5, 10, 20, 30, 40, 50, 60, 70, 80, 90, 95, 98, 99, or 100% by weight of the soft cartilage. According to another embodiment, at least a portion or all of the soft cartilage may be removed before cutting the samples. The bone marrow may then be removed. Non-limiting methods of removing bone marrow include soaking in Allowash® brand solution (LifeNet Health), aspiration, detergent (ionic, non-ionic and/or zwitterionic). The removal may be carried out in a processing chamber and may optionally include centrifugation in centrifuge and/or sonification in an ultrasonic chamber. Negative and/or positive pressure may be utilized, as may flowing and/or stirring of the cleaning solution. The step of removing the bone marrow also sterilizes the graft, since microbial population is also reduced. Removing the bone marrow also serves to reduce immunogenicity. The cartilage graft may be partially demineralized. Importantly, the demineralization is carried out only on a portion of the graft. In particular, the portion of the graft that is at least partially demineralized is the calcified cartilage, and optionally, at least a portion of the underlying subchondral bone plate. The demineralized portion of the graft thus provides the cartilaginous phase. The demineralization may be carried out on at least a portion of the subchondral bone plate and the calcified cartilage. Then, targeted growth factor removal may then be carried out. By “targeted” is meant that specific growth factors may be removed from the cartilaginous phase but not from the subchondral bone phase and/or that specific other growth factors may be removed from the subchondral bone phase but not from the cartilaginous phase. Controlled freezing may then be done as described above. The grafts may then be lyophilized as described above. When the graft is completed, it may be sealed into a package to maintain product sterility and stability. The packaged graft(s) may then be sterilized by gamma radiation or e-beam radiation. Other methods of sterilization include pasteurization, ethylene oxide, or solvent dehydration.

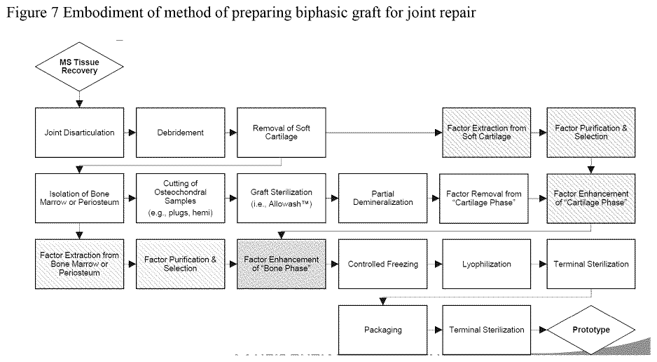

FIG. 7 shows another embodiment of a method of preparing a biphasic osteochondral graft according to the principles of the present invention. As shown in FIG. 7, this embodiment includes optional additional steps that are not shown in the FIG. 6 embodiment. These optional steps are depicted in FIG. 7 as follows.

Factor extraction may be performed on the soft cartilage, either before or after removing the soft cartilage from the graft. Preferably the factors are extracted from the graft cartilage after it is removed from the graft. Non-limiting examples of factors that may be removed from the soft cartilage may include bone morphogenetic proteins (BMPs), fibroblast growth factors (FGFRs), growth hormone (GH) insulin-growth factor I (IGF-I), transforming growth factor β (TGF-β), growth and differentiation factor 5 (GDF-5), glycosaminoglycans, such as hyaluronic acid, and extracellular vesicles (EVs). The factors may then be purified and if desired, separated. The factors, whether separated or not, may be added back to the cartilaginous phase in a factor enhancement step after the partial demineralization step and the step of targeted removal of certain other factors from the cartilaginous phase, if performed. If the removed factors are separated from each other, the desired factors may be selected and added to the cartilage phase in a factor enhancement step. According to another embodiment, the cartilaginous phase may be enriched with factors that are obtained from a source other than the soft cartilage that was removed from the source of the graft. The added factors may be natural or synthetic, if sourced from other than the soft cartilage removed from the source of the graft. As mentioned above, according to different embodiments, the source of the added factors may be cartilage from the donor of the graft, another donor and/or the recipient of the graft.

In an analogous manner, factors may be extracted from the bone marrow and/or periosteum and/or endosteum and/or bone matrix that are extracted from the subchondral bone in the bone phase. The factors may be extracted from bone. Non-limiting examples of factors that may be extracted from the bone marrow and or/periosteum and/or bone and/or endosteum and/or bone matrix are erythropoietin (EPO), granulocyte colony-stimulating factor (G-CSF), thrombopoietin (TPO), transforming growth factor-beta (TGF-β), insulin-like growth factors (IGF-1 and IGF-2), fibroblast growth factors (FGFs), platelet-derived growth factor (PDGF), bone morphogenetic proteins (BMPs), connective tissue growth factor (CTGF). The factors may then be purified and if desired, separated. The factors, whether separated or not, may be added back to the bone phase in a factor enhancement step after the partial demineralization step and the step of targeted removal of certain other factors from the bone phase, if done. If the removed factors are separated from each other, the desired factors may be selected and added to the bone phase in a factor enhancement step. According to another embodiment, the bone phase may be enriched with factors that are obtained from a source other than the bone marrow and/or periosteum that was removed from the source of the graft. The added factors may be natural or synthetic, if sourced from other than the bone marrow and/or periosteum and/or bone removed from the source of the graft. As mentioned above, according to different embodiments, the source of the added factors may be bone marrow and/or periosteum and/or endosteum and/or bone matrix and/or bone from the donor of the graft, another donor and/or the recipient of the graft. The added factors may be extracted and/or added to the bone phase according to the procedures disclosed in WO 2024/086171 A1.

In one embodiment, bone marrow may be removed from the bone phase of the graft, and the cartilaginous phase may be demineralized or partially demineralized, while the bone phase may be maintained to keep the cells viable. The processed graft may then then cryopreserved and stored before shipping to surgical facility for implantation.

In one embodiment, bone marrow may be removed from the bone phase, the whole graft may be cryopreserved and stored before shipping to surgical facility for implantation. The cells in the graft may maintain viability after cryopreservation and storage.

In another embodiment, drill holes may be generated in the bone phase to facilitate cell ingrowth and bony fusion. They may start from the bottom part, i.e., the end of the of the bone phase of biphasic graft that is the furthest from the interface between the bone phase and cartilaginous phase. The holes may end at or in the interface of the bone phase and cartilaginous phase, while not penetrating through the surface of the graft other than at their entry point at the bottom part, i.e., the end of the of the bone phase of biphasic graft that is the furthest from the interface between the bone phase and cartilaginous phase.

### Apparatus for Preparing a Biphasic Osteochondral Graft for Joint Repair

Also provided is an apparatus for performing the above method, i.e. for preparing the biphasic osteochondral graft for joint repair. The apparatus comprises the following components. A first chamber and a second chamber. The first chamber is constructed and arranged to contain the demineralization solution and/or the growth factor removal solution; and the cartilage-like (cartilaginous) phase. The second chamber is constructed and arranged to contain the buffering solution, and substantially non-demineralized subchondral bone phase. The first chamber and the second chamber are not in fluid communication.

At least one of the first and second chambers may each comprise a respective inlet and outlet such that the demineralization solution and/or the growth factor removal solution and/or the buffering solution can flow through each respective chamber.

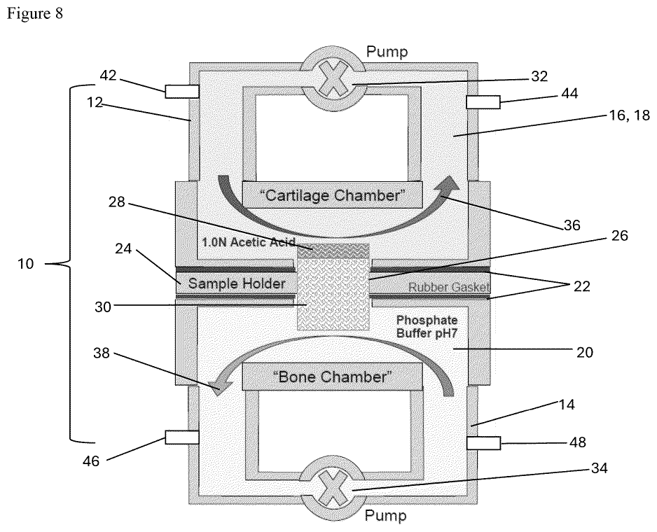

FIG. 8 shows a schematic of an embodiment of the apparatus for preparing the biphasic osteochondral graft. The apparatus is a dual-chambered targeted demineralization and targeted growth factor removal apparatus 10. As shown in FIG. 8, the dual-chamber apparatus 10 comprises a cartilage chamber 12 and a bone chamber 14. The cartilage chamber 12 contains the demineralization solution 16 and/or growth factor removal solution 18. The bone chamber 14 contains the buffer solution 20. The cartilage chamber 12 and the bone chamber 14 are not in fluid communication. A liquid-proof seal 22, which may be one or more rubber gaskets, is arranged between the cartilage chamber 12 and the bone chamber 14. A sample holder 24 is arranged to hold the biphasic osteochondral graft 26 in position such that the cartilaginous phase 28 is exposed to the demineralization solution 16 and/or the growth factor removal solution 18 contained in the cartilage chamber 12. The biphasic osteochondral graft 26 is held in position such that the substantially non-demineralized subchondral bone phase 30 is exposed to the buffer solution 20. The cartilage chamber 12 and the bone chamber 14 are each equipped with a respective pump 32, 34. The pumps 32 and 34 each provide fluid circulation in each respective chamber, as indicated by the arrows 36, 38. The cartilage chamber 12 may include an inlet 42 and outlet 44. Likewise, the bone chamber 14 may include an inlet 46 and outlet 48.

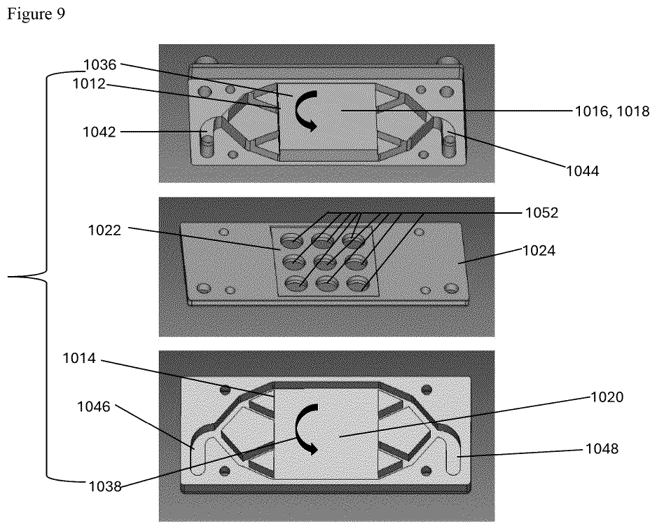

FIG. 9 shows an exploded view of another embodiment of a dual-chambered targeted demineralization and targeted growth factor removal apparatus 1010. As shown in FIG. 9, the dual-chamber apparatus 1010 comprises a cartilage chamber 1012 and a bone chamber 1014.

The cartilage chamber 1012 contains the demineralization solution 1016 and/or growth factor removal solution 1018. The bone chamber 1014 contains the buffer solution 1020. The cartilage chamber 1012 and the bone chamber 1014 are not in fluid communication. A liquid-proof seal, which may be one or more rubber gaskets 1022, is arranged between the cartilage chamber 1012 and the bone chamber 1014. A sample holder 1024 includes multiple openings 1052 to arranged to hold multiple biphasic osteochondral grafts (not shown) in position such that the cartilaginous phase is exposed to the demineralization solution 1016 and/or the growth factor removal solution 1018 contained in the cartilage chamber 1012. The biphasic osteochondral graft 1026 is held in position such that the substantially non-demineralized subchondral bone phase is exposed to the buffer solution 1020. The cartilage chamber 1012 and the bone chamber 1014 are each equipped with a respective pump 1032, 1034. Pumps (not shown), in or attached to each chamber, each provide fluid circulation in each respective chamber, as indicated by the arrows 1036, 1038. The cartilage chamber 1012 may include an inlet 1042 and outlet 1044. Likewise, the bone chamber 1014 may include an inlet 1046 and outlet 1048.

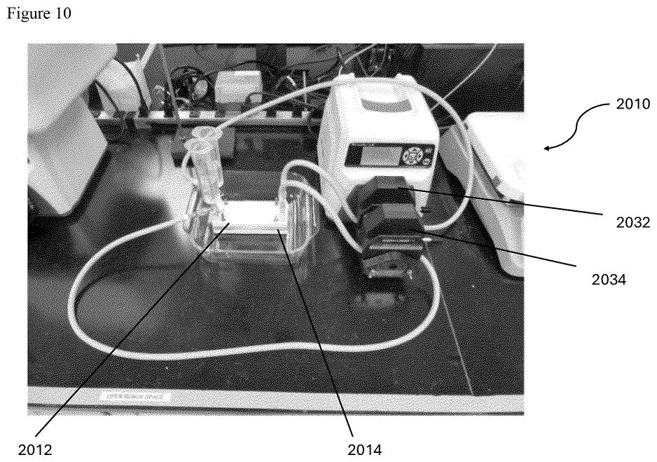

FIG. 10 shows a photograph of an embodiment of a dual-chambered targeted demineralization and targeted growth factor removal apparatus 2010 and pumps 2032 and 2034 associated with the cartilage chamber 2012 and bone chamber 2014, respectively.

### Method of Repairing an Osteochondral Defect

A method of repairing an osteochondral defect in a subject in need thereof is provided. The method comprises the following steps. A step of implanting at least one biphasic osteochondral graft into the osteochondral defect such that the substantially non-demineralized subchondral bone phase of the biphasic osteochondral graft is in an interior of the defect and the cartilage-like (cartilaginous phase) of the biphasic osteochondral graft is on an exterior of the defect may be done. Sealing the implanted biphasic osteochondral graft into recipient tissue may be done.

The method of repairing an osteochondral defect in a subject in need thereof may comprise shaping at least one biphasic osteochondral graft before implanting the shaped biphasic osteochondral graft(s) in the osteochondral defect. The shaping may be done by a scalpel, a Dremel, saws, files, drills, mallets, and/or scissors, for example. This shaped, i.e., size-matched graft may be placed in a confined osteochondral defect (OCD) lesion with conventional instrumentation (Osteochondral Allograft Transfer Systems [OATS], for example).

According to an embodiment, either before or after shaping, a lyophilized biphasic osteochondral graft for joint repair may be rehydrated with a biocompatible fluid, such as water, saline, blood or bone marrow-derived orthobiologics or a blood analog. Rehydration solutions may also contain bioactive factors. For example, the rehydration solutions may include general or targeted antimicrobial agents. The step of rehydration of the graft may be done with aqueous solutions (e.g., water for irrigation, saline) or patient-derived biologics (e.g., platelet-rich plasma [PRP] or bone marrow aspirate concentrate [BMAC]).

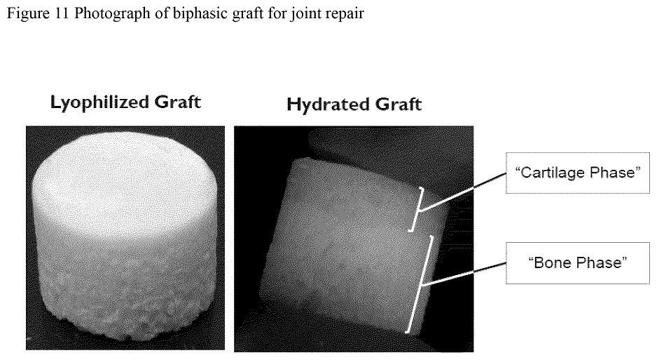

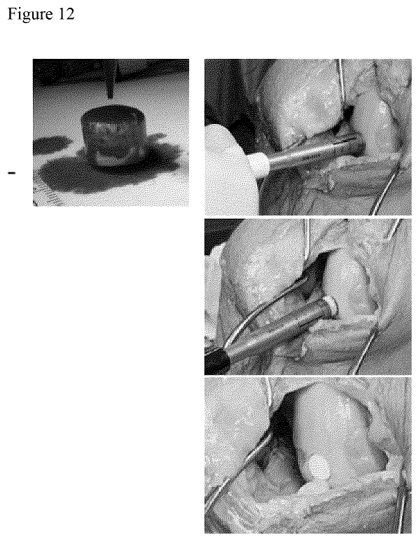

FIG. 11 shows photographs of lyophilized biphasic osteochondral graft for joint repair and a hydrated biphasic osteochondral graft for joint repair. FIG. 12 shows rehydration of a biphasic osteochondral graft for joint repair in blood analogue, and placement of a single biphasic graft using OATS instrumentation.

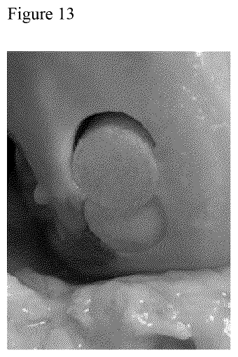

According to another embodiment, the method of repairing an osteochondral defect in a subject in need thereof two or more biphasic osteochondral grafts may be implanted in the osteochondral defect. Repairing the osteochondral defect may comprise performing a so-called snowman procedure. An example of a snowman procedure is shown in FIG. 13. As is known in the art, a snowman procedure involves inserting two or more overlapping grafts into the defect to be repaired. FIG. 13 shows how one of the two or more grafts may be shaped in the operating room to accept the contour of the other graft.

### EXAMPLES

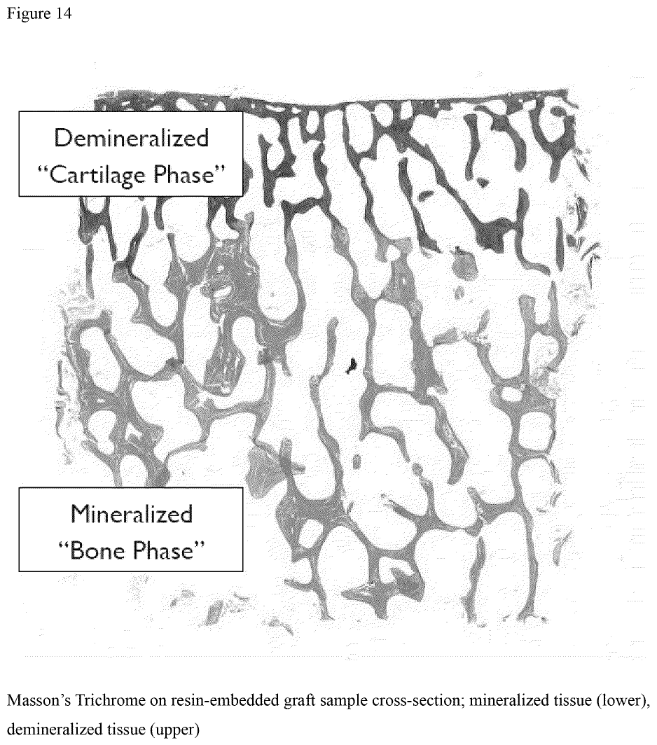

FIG. 14 shows a sample of a graft according to an exemplary embodiment of the present disclosure. The sample is a Masson's Trichrome on a resin-embedded graft sample cross-section. As shown, mineralized tissue is in the bone phase (lower), and demineralized tissue is in the cartilaginous phase (upper).

The lyophilized biphasic graft of FIG. 14 was prepared by being embedded in acrylic resin. Thin transverse sections were cut from each specimen and hand ground to a sample thickness of 5-7 μm. These samples were treated in sodium methoxide and rehydrated using decreasing ethanol solutions in phosphate buffer. Fully hydrated samples were sequentially stained with Masson's trichrome staining: celestine blue staining for 2-3 minutes, followed by hematoxylin staining for 2-3 minutes, Ponceau masson acid fuchsin staining for 10 minutes and aniline blue staining for 5 minutes at 37 °C. The samples were dehydrated in increasing ethanol solutions cleared in xylene and then mounted with a coverslip for imaging. The samples were imaged by light microscopy at low magnification to visualize the whole specimen and observe mineralized and demineralized graft portions as shown in the Figure.

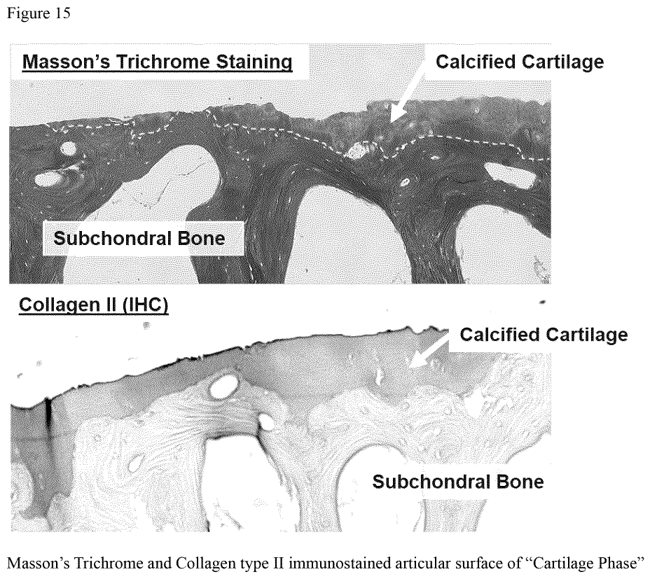

FIG. 15 shows Masson's Trichrome staining of a biphasic graft according to an exemplary embodiment of the invention. FIG. 15 also shows Collagen type II immunostained articular surface of the cartilaginous phase of a graft prepared according to an embodiment of the invention. These stained samples demonstrate the presence of calcified cartilage within the graft's cartilaginous phase and also show that the graft retained the presence of collagen in the calcified cartilage within the graft's cartilaginous phase.

The samples shown in FIG. 15 were prepared as follows. Fully hydrated samples were sequentially stained immunohistochemically to show the presence of Collagen type II. Samples were blocked for 60 minutes in 10% bovine serum albumin in phosphate buffer, incubated in a 1:100 biotinylated anti-human anti-Collagen II antibody at 4 °C overnight, incubated with a streptavidin conjugated horseradish peroxidase (HRP) and developed with Diaminobenzidine (DAB) to stain the antigens. Samples were sequentially counterstained in Hematoxylin and Eosin staining solutions, dehydrated in ethanol and mounted with a coverslip for imaging. The samples were analyzed for positive staining pattern at 10× magnification by light microscopy.

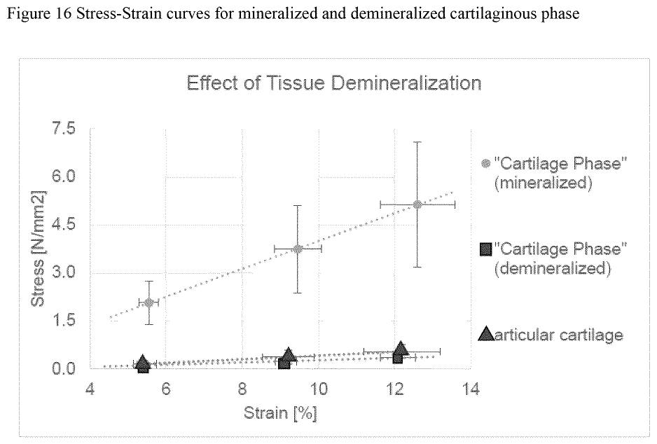

FIG. 16 shows stress-strain plots of the at least partially demineralized cartilaginous phase (i.e., after being subjected to the methods as described herein) compared to normal articular cartilage and also compared to non-demineralized cartilaginous phase (i.e., the section of the graft that becomes the cartilaginous phase prior to being subjected to the methods as described herein). The elastic properties of the at least partially demineralized cartilaginous phase were measured on rigid non-demineralized bone, i.e., the bone phase of the graft as described herein. As seen in FIG. 16, prior to being subjected to the methods as described herein, the section of the graft that becomes the non-demineralized cartilaginous phase has a higher modulus than the same cartilaginous phase of the biphasic graft for joint repair after demineralization. The non-demineralized cartilaginous phase (“cartilage phase”) is shown by the upper line (circles) in FIG. 16. Notably, the cartilaginous phase of the biphasic graft for joint repair after the demineralization step has a greatly reduced modulus and stress-strain response (lowest line-squares) when compared to the cartilage phase prior to demineralization, much like the soft cartilage that was removed from the source of the graft (middle line-triangles).

The data shown in FIG. 16 was obtained as follows. First, 5 mm high osteochondral segments were removed from the articulating surface of a human condyle. A segment of the articular sheet with soft articular cartilage was retained and served as an “articular cartilage” control, which is shown as the triangles in the FIG. 16 graph. To prepare the demineralized cartilaginous phase “cartilage phase” shown as squares in FIG. 16, this soft cartilage was exposed to 1.0 N HCl or phosphate buffer. The test samples were 10 mm in diameter. They were subjected to sequential indentation testing targeting 5, 8, and 10% maximum force strains.

Samples were kept hydrated in phosphate buffer during this unconfined testing using a 1 mm flat indenter and allowed to relax for 5 min prior to the ramp in strain. The samples' strain-stress characteristics were determined in triplicates for each test group and data is graphically in the strain-stress curves of FIG. 16. The unconstrained compressive modulus is the slope of the stress-strain curve. The method of measuring the stress-strain characteristics is described in Kabir et al. Cartilage 2021, Vol. 13 (Suppl 2) 427S-437S (11 pages), which is incorporated by reference herein.

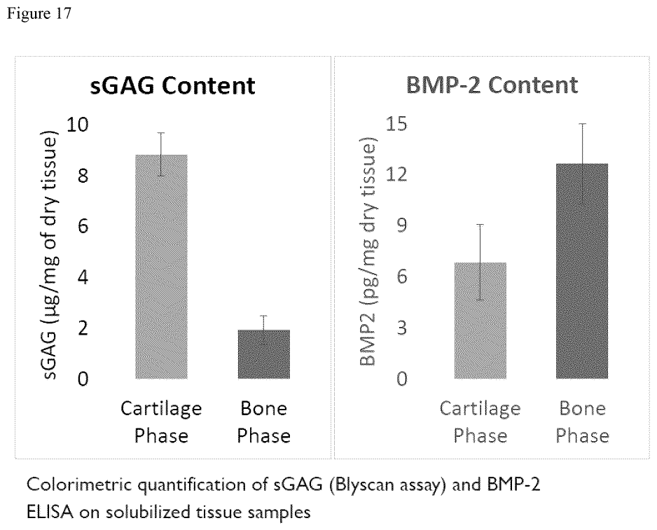

FIG. 17 shows colorimetric quantification of sGAG (Blyscan assay) and BMP-2 ELISA on solubilized tissue samples of the cartilaginous phase and the bone phase components of the biphasic grafts for joint repair according to some embodiments of the invention. These histograms demonstrate that the demineralized, lower modulus cartilaginous phase retained more of the glycosaminoglycans than the bone phase of the grafts. The plots also show how the bone phase of the biphasic graft for joint repair retained more BMP-2 than the demineralized cartilaginous phase.

The data shown in FIG. 17 was collected as follows. Lyophilized biphasic grafts, prepared according to an embodiment of the invention were rehydrated in phosphate buffer at room temperature overnight until fully rehydrated. The grafts' cartilaginous phase was separated from the underlying non-demineralized bone phase by sharp dissection. The non-demineralized mineralized bone phase was demineralized in 1.0 HCl overnight at room temperature under agitation and neutralized in sequential saline and sodium phosphate buffer rinses until the rinsate was neutral. Cartilage phase and demineralized bone phase were freeze-dried and solubilized overnight in collagenase buffer at 37 °C. The samples' sulfated glycosaminoglycan (sGAG) and bone morphogenetic protein-2 (BMP-2) content was determined using a commercial colorimetric detection system (Blyscan assay kit, Biocolor) or a BMP-2 detection kit (BMP-2 ELISA kit Quantikine, R&D Systems) as by the manufacturers' instructions.

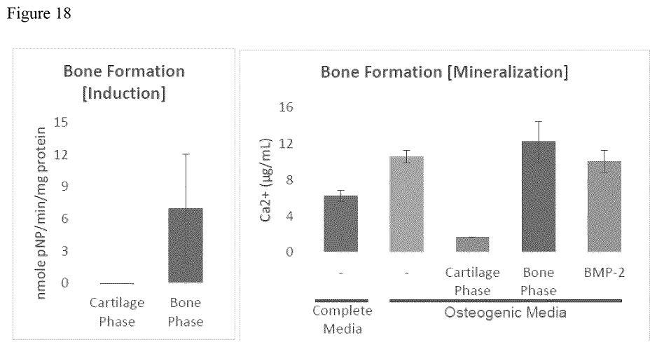

FIG. 18 shows an assessment of the osteoinductivity of both the bone phase and the cartilaginous phase of the osteochondral graft. The osteoinductivity was determined via Alkaline Phosphatase activity in C2C12 musculoskeletal progenitor cells in conditioned media. FIG. 18 also shows an assessment of phases of the grafts' mineralization capacity via quantification of tissue mineralization of bone marrow-MSCs in conditioned media. As shown in FIG. 18, the bone phase retained osteoinductivity, while the cartilaginous phase had almost no osteoinductivity. These important results show that these biphasic grafts will have desirable low mineralization in the cartilaginous phase while retaining the mineralization potential of the bone phase.

The data expressed in FIG. 18 was obtained as follows. The supplemented cell culture media to determine the osteogenicities of the cartilaginous phase and the non-demineralized bone phase were prepared by supplementing basic medium, DMEM containing 1% fetal calf serum (FCS), with 10 mg collagenase-solubilized tissue extracts. To assess the ability of extracts to induce osteogenesis, 50,000 C2C12 musculoskeletal progenitor cells were seeded onto 24 wells in growth medium, DMEM containing 10% FCS, and left to adhere overnight. Cells were rinsed and cultured for three days in supplemented media without media exchange. At the endpoint, the cells were lysed, and total protein and alkaline phosphate content were determined using a commercial Bicinchoninic Acid (BCA) Assay kit (Thermo Fisher) and Alkaline Phosphatase Assay Kit (Millipore-Sigma) as per manufacturers' instructions. Each material's osteoinductive potential was expressed as the accumulation of p-nitrophenol per minute per mg total protein content.

To assess the ability of extracts to support tissue mineralization which is shown in FIGS. 18, 50,000 human bone marrow-derived mesenchymal stem cells were seeded onto 24 wells in growth medium, DMEM containing 10% FCS, and left to adhere overnight. Cells were switched to osteogenic media supplemented with collagenase-solubilized cartilage phase or demineralized bone phase and culture for 21 days, changing the media every 2-3 days. At the endpoint, the cells were lysed in 0.5 N HCl and the samples' levels of tissue mineralization were determined by calcium content using a commercial calcium detection assay (Pointe Scientific) as per manufacturers' instructions.

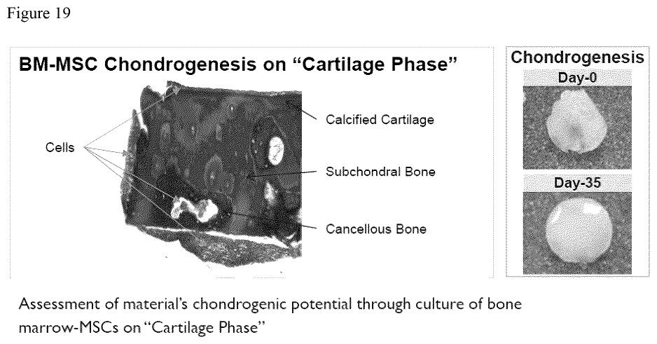

FIG. 19 shows an assessment of the graft's chondrogenic potential through culture of bone marrow-MSCs on “Cartilage Phase” (cartilaginous phase) component of the biphasic graft for joint repair. As shown in FIG. 19 the “Cartilage Phase” supported bone marrow-MSCs (mesenchymal stem cells) chondrogenic differentiation and cartilaginous neotissue formation in vitro.

The tissues shown in the FIG. 19 photographs were prepared as follows. First, 5 mm high osteochondral segments were removed from the articulating surface of a human condyle. The soft cartilage was removed by blunt resection. The resulting tissue sample was demineralized in 1.0 N HCl for 12 hours at room temperature and growth factors were removed in 4.0 M Guanidine HCl for 48 hours at 4 °C. Samples were rinsed, freeze-dried, and sterilized by gamma irradiation prior to use. Test samples were rehydrated for 30 minutes in growth medium prior to use. To assess chondrogenesis, 100,000 human bone marrow-derived mesenchymal stem cells were seeded directly onto the sample articular subchondral surface and continued in culture in osteogenic medium for three weeks. The appearance of the samples was observed macroscopically. To assess cartilaginous tissue formation, samples were fixed in 4% formalin for three days, paraffin embedded and evaluated histologically by Hematoxylin and Eosin and Safranin O staining and immunohistologically for the presence of Collagen II. The samples were 6 μm cross-sections which were prepared, dewaxed and rehydrated in decreasing ethanol solutions in phosphate buffer. Fully hydrated samples were sequentially stained with Hematoxylin and Eosin (Weigert's Iron Hematoxylin staining for 2-3 minutes, dilute Fast Green solution for 2-3 minutes at 37 °C) or Hematoxylin and Eosin with Safranin O staining. Fully hydrated samples were further sequentially stained immunohistochemically for the presence of Collagen type II. Samples were blocked for 60 minutes in 10% bovine serum albumin (BSA) in phosphate buffer, incubated in a 1:100 biotinylated anti-human anti-Collagen II antibody at 4 °C overnight, incubated with a streptavidin conjugated horseradish peroxidase (HRP) and developed with Diaminobenzidine (DAB) to stain antigens before being sequentially counterstained in Hematoxylin and Eosin staining solutions. All samples were dehydrated in increasing ethanol solutions and mounted with a coverslip. Samples were analyzed for staining pattern at 5× or 20× magnification by light microscopy.

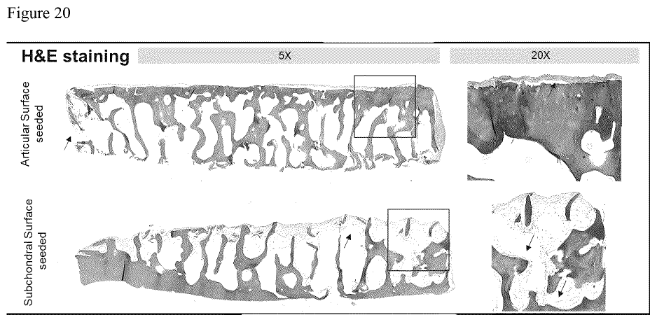

FIG. 20 shows Hematoxylin and Eosin staining of seeded articular surface (cartilaginous phase) and seeded subchondral surface (bone phase) at 5× and 20×. The results demonstrate that the cartilaginous phase supported bone marrow-MSCs chondrogenic differentiation and cartilaginous neotissue formation in vitro.

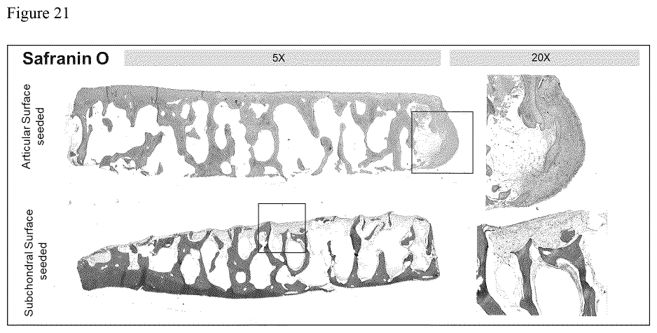

FIG. 21 shows Safranin O staining of seeded articular surface (cartilaginous phase) and seeded subchondral surface (bone phase) of the grafts as prepared according to an aspect of the method disclosed herein at 5× and 20×. The results demonstrate that the cartilaginous phase supported bone marrow-MSCs chondrogenic differentiation and cartilaginous neotissue formation in vitro as indicated by presence of GAGs (glycosaminoglycans).

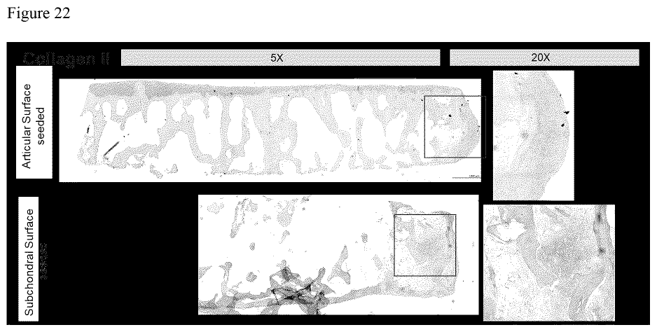

FIG. 22 shows how the cartilaginous phase supported bone marrow-MSCs chondrogenic differentiation and cartilaginous neotissue formation in vitro indicated by the presence of Collagen II (ICC) for seeded articular surface (cartilaginous phase) and seeded subchondral surface (bone phase) at 5× and 20×. These grafts were prepared according to an embodiment of the methods disclosed herein.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A unitary biphasic osteochondral graft for joint repair comprising:
at least two adjacent contacting phases:
a cartilaginous phase comprising at least a portion of a subchondral bone plate, wherein at least a portion of the subchondral bone plate is partially demineralized; and
a substantially non-demineralized subchondral bone phase.

2. The biphasic osteochondral graft for joint repair of claim 1, wherein:
a modulus of the cartilaginous phase is less than a modulus of the substantially non-demineralized subchondral bone phase.

3. The biphasic osteochondral graft for joint repair of claim 1, wherein the cartilaginous phase further comprises an at least partially demineralized calcified cartilage layer.

4. The biphasic osteochondral graft for joint repair of claim 1, wherein the bone phase remains viable.

5. The biphasic osteochondral graft for joint repair of claim 1, wherein the cartilaginous phase comprises less than 45 pg/mg dry tissue of bone morphogenetic protein 2 (BMP-2).

6. The biphasic osteochondral graft for joint repair of claim 1, wherein the substantially non-demineralized subchondral bone phase comprises 10 pg/mg dry tissue or more of bone morphogenetic protein 2 (BMP-2).

7. The biphasic osteochondral graft for joint repair of claim 1, wherein the biphasic osteochondral graft for joint repair is capable of being shaped.

8. A method of preparing a unitary biphasic osteochondral graft for joint repair comprising:
obtaining a joint from a donor, wherein the joint comprises a subchondral bone plate and subchondral bone;
at least partially demineralizing at least a portion of the subchondral bone plate with a demineralization solution to provide the unitary biphasic osteochondral graft wherein the unitary biphasic osteochondral graft comprises:
a cartilaginous phase comprising the demineralized subchondral bone plate which was at least partially demineralized; and
a substantially non-demineralized subchondral bone phase comprising any remaining portion of non-demineralized subchondral bone plate and non-demineralized subchondral bone.

9. The method of claim 8, wherein the cartilaginous phase further comprises calcified cartilage, and
wherein the demineralizing step further comprises partially demineralizing at least a portion of the calcified cartilage; and wherein the cartilaginous phase further comprises the demineralized calcified cartilage.

10. The method of claim 8, wherein a modulus of the cartilaginous phase is less than a modulus of the non-demineralized calcified cartilage.

11. The method of claim 8, wherein a modulus of the cartilaginous phase is less than a modulus of the non-demineralized subchondral bone.

12. The method of claim 8, wherein a modulus of the substantially non-demineralized bone phase is about the same as a modulus of the non-demineralized subchondral bone.

13. The method of claim 8, further comprising a step of exposing at least a portion of the subchondral bone plate to a buffering solution, to provide the substantially non-demineralized bone phase, wherein the exposing is performed simultaneously with or after the demineralization step.

14. The method of claim 8, further comprising a step of exposing at least a portion of the subchondral bone to a buffering solution, to provide the substantially non-demineralized bone phase, wherein the exposing is performed simultaneously with or after the demineralization step.

15. The method of claim 8, further comprising removing bone morphogenetic protein 2 (BMP-2) from the cartilaginous phase with a growth factor removal solution, without substantially removing bone morphogenetic protein 2 (BMP-2) from the substantially non-demineralized bone phase.

16. The method of claim 8, wherein cells in the bone phase of the biphasic osteochondral graft remain viable.

17. A method of repairing an osteochondral defect in a subject in need thereof, comprising:
implanting at least one biphasic osteochondral graft of claim 1 into the osteochondral defect such that the substantially non-demineralized subchondral bone phase of the biphasic osteochondral graft is in an interior of the defect and the cartilaginous phase of the biphasic osteochondral graft is on an exterior of the defect; and
sealing the implanted biphasic osteochondral graft into recipient tissue.

18. The method of repairing an osteochondral defect in a subject in need thereof of claim 17, comprising:
shaping the at least one biphasic osteochondral graft before implanting the shaped biphasic osteochondral graft(s) in the osteochondral defect.

19. The method of repairing an osteochondral defect in a subject in need thereof of claim 17, wherein two or more biphasic osteochondral grafts are implanted in the osteochondral defect and wherein the repairing comprises performing a snowman procedure.

20. An apparatus for performing the method of claim 8 comprising:
a first chamber and a second chamber, wherein
the first chamber is constructed and arranged to contain the demineralization solution and/or the growth factor removal solution, and the cartilaginous phase;
the second chamber is constructed and arranged to contain the buffering solution, and substantially non-demineralized subchondral bone phase;
and wherein the first chamber and the second chamber are not in fluid communication.

21. The apparatus of claim 20, wherein at least one of the first and second chambers each comprise a respective inlet and outlet such that the demineralization solution and/or the growth factor removal solution and/or the buffering solution can flow through each respective chamber.
